(19) United States
(12) Patent Application Publication
(10) Pub. No.: US 2004/0102647 A1
(43) Pub. Date: May 27, 2004

(54) Acetaldehyde dehydration to produce ethyne

(76) Inventor: Christian Everett, Springfield, IL (US)

Correspondence Address: CHRISTIAN EVERETT, 2743 SOUTH VETERANS PKWY #302, SPRINGFIELD, IL, 62704, US

(21) Appl. No.: 10/673,946

(22) Filed: Sep. 30, 2003

Related U.S. Application Data
(63) Continuation-in-part of application No. 09/910,248, filed on Jul. 20, 2001, now abandoned.
(60) Provisional application No. 60/228,828, filed on Aug. 28, 2000.

(51) Int. Cl. C07C 5/32; C07C 51/235

(52) U.S. Cl. 562/532; 585/534

(57) ABSTRACT

A process to produce ethyne comprising passing acetaldehyde in the gas phase through a reaction zone containing a dehydrating metal oxide catalyst such as aluminum oxide or magnesium oxide at a temperature of approximately 375 C. to produce ethyne and water and a cooling zone following the reaction zone.

This method having the advantage of ease of separation of ethyne from the co-product water and unreacted acetaldehyde by simple condensation whereby the acetaldehyde and water liquify while The ethyne remains gaseous.

### BACKGROUND-FIELD OF INVENTION

This invention relates to the production of ethyne (acetylene) and substituted ethynes.

### BACKGROUND-DESCRIPTION OF PRIOR ART

Ethyne has been produced by the high temperature pyrolisis of ethene (U.S. Pat. No. 5,942,653) and methane, which is currently the industry standard. The disadvantages of this process is the high reaction temperature (1200 C.), low yields (30%) and the difficulty of separating the ethyne from the reactant methane and co-product hydrogen which results in the need for an elaborate process whereby the gasses are isolated by their different solubilities in various organic liquids. Also the reaction must be immediately quenched from 1200 C. to 300 C. to preserve the ethyne that is formed. all this contributes to the retail price of ethyne being $ 6 per pound ($36 per gallon 6/2000).

### OBJECTS AND ADVANTAGES

Several objects and advantages of the present invention are:

(A) Lower reaction temperature.

(B) Higher yield.

(C) Ease of separation of product ethyne from reactant acetaldehyde and co-product water.

(D) Low cost feedstock acetaldehyde (26 cents per pound 6/2000) which is itself produced from ethanol.

(E) Reaction is specific to aldehyde group allowing production of substituted ethynes from substituted acetaldehyde compounds up to ten carbon atoms, which is not possible with pyrolisis or partial oxidation processes.

### SUMMARY

In accordance with the present invention a process to produce ethyne and substituted ethynes by the dehydration of acetaldehyde and substituted acetaldehyde compounds whereby a molecule of water is removed from each acetaldehyde moiety by passing the compound through a dehydrating metal oxide catalyst at an elevated temperature, then cooling the resulting gas stream to condense unreacted acetaldehyde and co-product water whereby ethyne is obtained as a gas.

### DESCRIPTION

The formation of the carbon-carbon triple bond characteristic of ethyne is thermodynamically unfavorable, as it possesses a large positive heat of formation (+530 Kcals/mole). This large quantity of energy is retrieved when the molecule is decomposed, for example by oxidation during burning as a welding gas. The energy to form the triple bond may be supplied by the electric arc process, which provides energy in the form of heat, or by a chemical process such as the reaction of a vicinal di-chloride with sodium hydroxide. In the chemical process the production of sodium chloride which has a large negative heat of formation (−750 Kcals/mole) makes the overall reaction to form co-product ethyne thermodynamically favorable (exothermic), and the reaction occurs at ordinary temperatures (100 C.).

In the dehydration of aldols such as acetaldehyde the formation of water, which has a large negative heat of formation (−480 Kcals/mole) as a co-product makes the reaction, although still endothermic, considerably more favorable than pyrolysis of hydrocarbons where the co-product hydrogen has a heat of formation of zero. Therefore less energy in the form of heat is required to drive the reaction, and the reaction proceeds at a lower temperature.

The most thermodynamically favorable temperature for the dehydration reaction of acetaldehyde to ethyne and water is about 600 C. with a delta H of about +80 Kcals/mole, or about the same as the formation of nitric oxide from nitrogen and oxygen or twice that of ammonia from nitrogen and hydrogen. However, acetaldehyde decomposes at temperatures above 400 C. forming principally methane and carbon monoxide. The activation energy of the decomposition reaction is 408 Kcals/mole.

The dehydration catalyst is selected from a group of metal oxides that possess dehydrating properties, aluminum oxide being the preferred catalyst as it is 100% dehydrating. Magnesium oxide is second as it is 91% dehydrating and 9% dehydrogenating.

Ethyne boils at −84 C., but ethyne produced by pyrolisis cannot be separated from methane (b.p. −161 C.) and hydrogen (b.p. −250 C.) by condensation because it cannot be liquefied safely since it is thermodynamically unstable and explodes even in the absence of oxygen to form carbon and hydrogen. In the dehydration reaction process co-effluents acetaldehyde (b.p. 15 C.) and water (b.p. 100 C.) are condensed, leaving the ethyne as a gas.

### EXAMPLE 1

Acetaldehyde in the gas phase is passed through a reaction zone containing aluminum oxide at 375 C. to produce ethyne and water. The gas stream is cooled to 100 C. by passing through a cooling zone. The gas stream is then chilled to 0 C. causing the co-product water and unreacted acetaldehyde to liquefy whereby ethyne containing some acetaldehyde vapor is obtained as a gas. The ethyne may be further purified by reducing the temperature to −50 C., whereby the vapor pressure of the acetaldehyde is reduced to 18 mmHg leaving the ethyne 98% pure:

### EXAMPLE 2

Propionaldehyde in the gas phase is passed through a reaction zone containing aluminum oxide at 375 C. to produce propyne (isomeric with propadiene) and water. The gas stream is cooled to 100 C. by passing through a cooling zone. The gas stream is then chilled to 0 C. causing the co-product water and unreacted propionaldehyde to liquify whereby propyne containing some propionaldehyde vapor is obtained as a gas. The propyne may be further purified by reducing the temperature to −35 C., whereby the vapor pressure of the propionaldehyde is reduced to 20 mmHg, leaving the propyne 98% pure.

### EXAMPLE 3

Succindialdehyde in the gas phase is passed through a reaction zone containing aluminum oxide at a temperature of 350 C. to produce diacetylene and water. The gas stream is cooled to 100 C. by passing through a cooling zone. The gas stream is then chilled to 10 C. causing the co-product water and unreacted or partially reacted succindialdehyde to liquify, whereby diacetylene is obtained as a gas.

### EXAMPLE 4

Acetone in the gas phase is passed through a reaction zone containing aluminum oxide at 400 C. to produce propyne (isomeric with propadiene) and water. The gas stream is then cooled to 100 C. by passing through a cooling zone. The gas stream is then chilled to 0 C. causing the co-product water and unreacted acetone to condense whereby propyne containing some acetone vapor is obtained as a gas. The propyne may by further purified by reducing the temperature to −35, whereby the vapor pressure of the acetone is reduced to 15 mmHg, leaving the propyne 98% pure.

### CONCLUSION, RAMIFICATION AND SCOPE

Accordingly, the reader will see the production of ethyne by the dehydration of acetaldehyde is a more convenient process than current methods. Furthermore the process has additional advantages in that:

Ultimate feedstock ethanol can be produced from renewable resources.

Production plant need not be located at methane production site as acetaldehyde and ethanol can be shipped by tank truck or rail, which methane cannot.

Ultimate cost of ethyne will be less than by existing methods

Reaction is general for aldehydes and ketones and may be used to produces substituted ethynes.

Although the description contains specificities, these should not be construed as limiting the scope of the invention, for example higher or lower temperatures may be used and other aldehydes and ketones may be used. Thus the scope of the invention should be determined by the appended claims and their legal equivalents rather than by the examples given.

I claim:

1) a method for producing ethyne, comprising the steps of
(a) passing acetaldehyde in the gas phase through a reaction zone containing a dehydrating metal oxide catalyst such as aluminum oxide or magnesium oxide at a temperature of from approximately 250 C. to approximately 400 C. to remove a molecule of water from the reactant acetaldehyde to produce ethyne and co-product water:
(B) Cooling the resulting gas stream of ethyne, co-product water and unreacted acetaldehyde to a temperature of approximately −50 C. to condense the water and acetaldehyde whereby the ethyne is obtained as a gas.

2) A method for producing substituted ethynes, comprising the steps of
(A) Passing a mono-alpha substituted acetaldehyde, where one hydrogen on the carbon alpha to the aldehyde group is replaced by an aryl or alkyl group which may include up to three additional mono-alpha substituted acetaldehyde moieties, up to a total of ten carbon atoms, including propionaldehyde, succindialdehyde and phenylacetaldehyde through a reaction zone containing a dehydrating metal oxide catalyst such as aluminum oxide or magnesium oxide at a temperature of from approximately 250 C. to approximately 400 C. to remove a molecule of water from each acetaldehyde moiety in the reactant to produce substituted ethyne and co-product water.
(B) Cooling the resulting gas stream of substituted ethyne, co-product water and unreacted or partially reacted aldehyde and separating the product substituted ethyne from the co-product water and unreacted or partially reacted aldehyde by a physical process such as fractional distillation, fractional crystallization or dessication or a chemical process such as forming an imide or acetal or bisulfite of the aldehyde, or a combination of physical and chemical methods whereby the substituted ethyne is obtained.

3) A method for producing substituted ethynes, comprising the steps of
(A) Passing a carbonyl hydrogen substituted acetaldehyde, where the hydrogen of the aldehyde group is replaced by a substituent which is either fluorine or chlorine, an ether linkage or or an aryl or alkyl group which may contain up to three additional carbonyl hydrogen or mono-alpha substituted acetaldehyde moieties up to a total of ten carbon atoms, these compounds including acetyl chloride, acetic anhydride, ethyl acetate and acetone through a reaction zone containing a dehydrating metal oxide catalyst such as aluminum oxide or magnesium oxide at a temperature of approximately 250 C. to approximately 450 C. to remove a molecule of water from each acetaldehyde moiety to produce substituted ethyne and co-product water.
(B) Cooling the resulting gas stream of substituted ethyne, co-product water and unreacted or partially reacted substituted acetaldehyde and separating the product substituted ethyne from the co-product water and unreacted or partially reacted substituted acetaldehyde by a physical process such as fractional distillation, fractional crystallization or desiccation or a chemical process such as forming an imide or acetal or bisulfite of the unreacted or partially reacted subsituted acetaldehyde or a combination of physical and chemical methods whereby the substituted ethyne is obtained.
